(19) United States
(12) Patent Application Publication
(10) Pub. No.: US 2003/0049698 A1
(43) Pub. Date: Mar. 13, 2003

(54) Diagnosis and treatment of gastrointestinal disease

(76) Inventor: Timothy C. Wang, Acton, MA (US)

Correspondence Address: WOLF GREENFIELD & SACKS, PC, FEDERAL RESERVE PLAZA, 600 ATLANTIC AVENUE, BOSTON, MA, 02210-2211, US

(21) Appl. No.: 10/257,169

(22) PCT Filed: Mar. 30, 2001
(86) PCT No.: PCT/US01/10259

(51) Int. Cl. G01N 33/567; G01N 33/554; G01N 33/569

(52) U.S. Cl. 435/7.21; 435/7.32

(57) ABSTRACT

The invention relates to methods for the diagnosis and treatment of gastrointestinal disease. More specifically, the invention relates to methods for the diagnosis and treatment of duodenal ulcer disease and gastric atrophy.

### GOVERNMENT SUPPORT

This work was funded in part by grant numbers R01-DK52778 and R01-CA67529 from the National Institutes of Health. Accordingly, the United States Government may have certain rights to this invention.

### FIELD OF THE INVENTION

The invention relates to methods for the diagnosis and treatment of gastrointestinal disease. More specifically, the invention relates to methods for the diagnosis and treatment of duodenal ulcer disease and gastric atrophy.

### BACKGROUND OF THE INVENTION

Gastrin is a peptide hormone synthesized in the stomach that stimulates acid secretion through direct action on parietal cells. Gastrin is initially synthesized as progastrin (nonamidated form), which is processed to glycine-extended gastrin (e.g., G-17-Gly, G-34-Gly, or G-Gly: nonamidated forms), and then to amidated gastrin (G-17 and G-34; amidated forms). The role of amidated gastrins has been the primary focus of research in the art involving Helicobacter pylori (H. pylori) as a disease causative agent.

It is now well established that H. pylori is a causative agent for both peptic ulcer disease (duodenal ulcer disease) and gastric-atrophy/gastric-cancer. Scientists have long been perplexed as to how one causative agent can lead to the development of two such distinct clinical outcomes. Various studies have reported on the possible links between H. pylori infection and an elevation in amidated gastrin (G-17) levels. G-17 levels are, reportedly, slightly higher in ulcer patients compared to non-ulcer patients. More recent studies, however, report that elevated G-17 levels are not predictive of an ulcer phenotype. In addition, G-17 levels are, reportedly, elevated in patients who develop gastric atrophy and/or gastric cancer.

A need exists for the diagnosis and therapy of gastrointestinal disease.

### SUMMARY OF THE INVENTION

This invention describes, in part, new diagnostic tests which can be predictive of gastrointestinal disease. These new tests broadly include: (i) the prediction of risk of future gastrointestinal disorders such as duodenal ulser disease and gastric atrophy; and (ii) the determination of the likelihood that certain individuals will benefit to a greater or lesser extent from the use of certain treatments designed to prevent and/or treat gastrointestinal disorders. These new tests are based in part upon the following discoveries.

It has been discovered that incompletely processed forms of gastrin molecules are predictive of gastrointestinal disease. More specifically, it has been discovered, unexpectedly, that in an individual with elevated levels of total gastrin, the ratio of amidated to non-amidated gastrins (of their respective body fluid concentrations), is predictive of the risk for developing duodenal ulcer disease versus gastric-atrophy/gastric-cancer.

It has been discovered also that the likelihood that certain individuals will benefit to a greater or a lesser extent from the use of certain therapeutic agents for reducing the risk of a gastrointestinal disease can be determined from the ratio of amidated to non-amidated gastrins in an individual.

It further has been discovered that certain individuals with a gastrointestinal disease or at risk of developing a gastrointestinal disease may benefit from the administration of incompletely processed forms of gastrin molecules.

According to one aspect of the invention, a method for characterizing an individual's risk profile of developing duodenal ulcer disease, is provided. The method involves obtaining a ratio of nonamidated gastrins to amidated gastrins in an individual with an elevated total gastrin level, comparing the ratio of nonamidated gastrins to amidated gastrins to a predetermined value, and characterizing the individual's risk profile of developing duodenal ulcer disease based upon the ratio of nonamidated gastrins to amidated gastrins in comparison to the predetermined value. The predetermined value can be a single value, multiple values, a single range or multiple ranges. Thus, in one embodiment, the predetermined value is a plurality of predetermined value ranges and the comparing step comprises determining in which of the predetermined value ranges the individual's ratio of nonamidated gastrins to amidated gastrins falls. In some embodiments, the predetermined value can be between about 1.1 and about 1.75, or higher. In further embodiments, the individual has not been previously suspected of having duodenal ulcer disease. In yet further embodiments, the individual has positive H. Pylori serology. In certain embodiments, the individual is asymptomatic for duodenal ulcer disease.

According to another aspect of the invention, a method for characterizing an individual's risk profile of developing gastric atrophy leading to gastric cancer, is provided. The method involves obtaining a ratio of nonamidated gastrins to amidated gastrins in an individual with an elevated total gastrin level, comparing the ratio of nonamidated gastrins to amidated gastrins to a predetermined value, and characterizing the individual's risk profile of developing gastric atrophy leading to gastric cancer, based upon the ratio of nonamidated gastrins to amidated gastrins in comparison to the predetermined value. The predetermined value can be a single value, multiple values, a single range or multiple ranges. Thus, in one embodiment, the predetermined value is a plurality of predetermined value ranges and the comparing step comprises determining in which of the predetermined value ranges the individual's ratio of nonamidated gastrins to amidated gastrins falls. In certain embodiments, the predetermined value can be below 0.9, preferably between about 0.9 and about 0.4, or lower. In some embodiments, the individual has positive H. Pylori serology. In further embodiments, the individual is a subject that receives proton-pump inhibitor treatment (e.g., to treat gastroesophageal reflux disease/“heartburn”).

According to another aspect of the invention, a method for evaluating the likelihood that an individual with an elevated total gastrin level will benefit from treatment with an agent useful in treating duodenal ulcer disease, is provided. The method involves obtaining a ratio of nonamidated gastrins to amidated gastrins in the individual, comparing the ratio of nonamidated gastrins to amidated gastrins to a predetermined value, wherein the ratio of nonamidated gastrins to amidated gastrins in comparison to the predetermined value is indicative of whether the individual will benefit from treatment with said agent, and characterizing whether the individual is likely to benefit from said treatment based upon said comparison. The predetermined value can be a single value, multiple values, a single range or multiple ranges. Thus, in one embodiment, the predetermined value is a plurality of predetermined value ranges and the comparing step comprises determining in which of the predetermined value ranges the individual's ratio of nonamidated gastrins to amidated gastrins falls. In certain embodiments, the predetermined value can be between about 1.1 and about 1.75, or higher than 1.75. In further embodiments, the individual has not been previously suspected of having duodenal ulcer disease. In yet further embodiments, the individual has positive H. Pylori serology. In certain embodiments, the individual is asymptomatic for duodenal ulcer disease. Preferred agents useful in treating duodenal ulcer disease include antacids, H-2 receptor antagonists, anticholinergic agents, coating agents, prostaglandins, proton-pump inhibitors, antibiotics, and/or combinations thereof.

According to another aspect of the invention, a method for evaluating the likelihood that an individual with an elevated total gastrin level will benefit from treatment with an agent useful in treating gastric atrophy, is provided. The method involves obtaining a ratio of nonamidated gastrins to amidated gastrins in the individual, comparing the ratio of nonamidated gastrins to amidated gastrins to a predetermined value, wherein the ratio of nonamidated gastrins to amidated gastrins in comparison to the predetermined value is indicative of whether the individual will benefit from treatment with said agent, and characterizing whether the individual is likely to benefit from said treatment based upon said comparison. The predetermined value can be a single value, multiple values, a single range or multiple ranges. Thus, in one embodiment, the predetermined value is a plurality of predetermined value ranges and the comparing step comprises determining in which of the predetermined value ranges the individual's ratio of nonamidated gastrins to amidated gastrins falls. In certain embodiments, the predetermined value can be below 0.9, preferably between about 0.9 and about 0.4, or lower. In some embodiments, the individual has positive H. Pylori serology. In certain embodiments, the individual is asymptomatic for gastric atrophy disease. In further embodiments, the individual is a subject that receives proton-pump inhibitor treatment. The agent useful in treating gastric atrophy includes a cholecystokini-B (CCK-B)/gastrin receptor antagonist, a proton-pump inhibitor, a nonamidated gastrin, and/or a combination thereof. Preferred nonamidated gastrins include G-Gly and progastrin. In an important embodiment, the agent useful in treating gastric atrophy is a combination of a proton-pump inhibitor and a CCK-B/gastrin receptor antagonist. In one embodiment, the molar ratio of the proton-pump inhibitor and the CCK-B/gastrin receptor antagonist is 1:1. In further embodiments, the molar ratio of the proton-pump inhibitor and the CCK-B/gastrin receptor antagonist can be between about 2:1 and at least about 30:1. In yet further embodiments, the molar ratio of the proton-pump inhibitor and the CCK-B/gastrin receptor antagonist can be between about 3:1 and at least about 30:1.

As mentioned above, the invention is particularly adapted to determining which individuals will preferentially benefit from treatment with an agent for reducing the risk of developing a gastrointestinal disorder. It also permits selection of candidate populations for clinical trials and for treatment with candidate drugs, by identifying, for example, the individuals most likely to benefit from a new treatment or from a known treatment with a high risk profile of adverse side effects. Thus, the invention provides information for evaluating the likely net benefit of certain treatments for candidate patients.

According to a further aspect of the invention, a method for treating an individual at risk of developing duodenal ulcer disease, is provided. The method involves selecting and administering to an individual having an elevated total gastrin level and an above-normal ratio of nonamidated gastrins to amidated gastrins, an agent useful in treating duodenal ulcer disease, in an amount effective to inhibit development of duodenal ulcer disease in the individual. In certain embodiments, the above-normal ratio of nonamidated gastrins to amidated gastrins can be between about 1.1:1 and about 1.75:1, or higher than 1.75:1. In some embodiments, the individual has positive H. Pylori serology. In certain embodiments, the individual is asymptomatic for duodenal ulcer disease. Preferred agents useful in treating duodenal ulcer disease include antacids, H-2 receptor antagonists, anticholinergic agents, coating agents, prostaglandins, proton-pump inhibitors, antibiotics, and/or combinations thereof.

According to yet another aspect of the invention, a method for treating an individual at risk of developing gastric atrophy, is provided. The method involves selecting and administering to an individual having an elevated total gastrin level and a below-normal ratio of nonamidated gastrins to amidated gastrins an agent that includes a CCK-B/gastrin receptor antagonist, a proton-pump inhibitor, a nonamidated gastrin, and/or a combination thereof, in an amount effective to inhibit development of gastric atrophy (parietal cell loss) in the individual. In one important embodiment, the individual is a subject that receives proton-pump inhibitor treatment. In another important embodiment of this aspect, the individual is asymptomatic for gastric atrophy. In further embodiments, the individual has gastric atrophy. In still further embodiments, the individual has positive H. Pylori serology. In certain embodiments, the individual is asymptomatic for duodenal ulcer disease. In still further important embodiments, the below-normal ratio of nonamidated gastrins to amidated gastrins can be between about 0.9:1 and about 0.4:1, or lower than 0.4:1. Preferred nonamidated gastrins include G-Gly and progastrin. Any of the foregoing agents useful in this aspect of the invention can be administered prophylactically or acutely. In an important embodiment, the agent useful in treating gastric atrophy is a combination of a proton-pump inhibitor and a CCK-B/gastrin receptor antagonist. In one embodiment, the molar ratio of the proton-pump inhibitor and the CCK-B/gastrin receptor antagonist is 1:1. In further embodiments, the molar ratio of the proton-pump inhibitor and the CCK-B/gastrin receptor antagonist can be between about 2:1 and at least about 30:1. In yet further embodiments, the molar ratio of the proton-pump inhibitor and the CCK-B/gastrin receptor antagonist can be between about 3:1 and at least about 30:1.

In another aspect, the invention provides a pharmaceutical preparation comprising at least one agent selected from the group consisting of a CCK-B/gastrin receptor antagonist, a proton-pump inhibitor, and a nonamidated gastrin, in an effective amount to inhibit development of gastric atrophy (parietal cell loss) in an individual, and a pharmaceutically-acceptable carrier. Preferred nonamidated gastrins include G-Gly and progastrin.

In an important embodiment, the pharmaceutical preparation comprises at least a proton-pump inhibitor and a CCK-B/gastrin receptor antagonist. In one embodiment, the molar ratio of the proton-pump inhibitor and the CCK-B/gastrin receptor antagonist is 1:1. In further embodiments, the molar ratio of the proton-pump inhibitor and the CCK-B/gastrin receptor antagonist can be between about 2:1 and at least about 30:1. In yet further embodiments, the molar ratio of the proton-pump inhibitor and the CCK-B/gastrin receptor antagonist can be between about 3:1 and at least about 30:1.

The invention also contemplates kits comprising a package including assays for detecting and quantitating amidated and non-amidated gastrins and instructions, and optionally related materials such as number or color charts, for correlating the level of gastrin ratios as determined by the assays with a predetermined value to evaluate the risk of developing a future gastrointestinal disorder, or with other patient criteria as described above. In important embodiments, the kits also include an assay for H. pylori infection.

The invention also contemplates software that is based on the ratios of amidated and non-amidated gastrins and their comparison to predetermined values to calculate the risk of an individual developing gastrointestinal disease.

These and other aspects of the invention, as well as various advantages and utilities, will be more apparent with reference to the detailed description of the preferred embodiments.

### BRIEF DESCRIPTION OF THE DRAWINGS

FIG. 1 depicts graphs showing that in a transgenic mouse model of hypegastrinemia (INS-GAS), loss of initial gastric acid hypersecretion and increasing serum gastrin levels over time are observed; (A) Serum gastrin levels in INS-GAS mice over time; (B) Gastric acid output; (C) Parietal cell counts over time; and (D) ECL cell number in wild type and INS-GAS mice at 1 and 12 months of age.

FIG. 2 depicts graphs showing that G17-Gly augments G-17 induced acid secretion in gastrin deficient mice.

FIG. 3 depicts a kit comprising agents of the invention (e.g., anti-gastrin specific antibodies, gastrin epitopes, etc.), and instructions for utilizing such agents in diagnostic or therapeutic applications.

### DETAILED DESCRIPTION OF THE INVENTION

The invention involves the discovery that incompletely processed forms of gastrin molecules play a predictive role in the development of gastrointestinal disease. The invention is based on the unexpected discovery that the ratio of amidated to non-amidated gastrins (of their respective serum concentrations), is predictive of the risk for developing duodenal ulcer disease versus gastric-atrophy/gastric-cancer in an individual with elevated levels of total gastrin.

It has been discovered also that the likelihood that certain individuals will benefit to a greater or a lesser extent from the use of certain therapeutic agents for reducing the risk of a gastrointestinal disease can be determined from the ratio of amidated to non-amidated gastrins in an individual.

As used herein, the term “individual” is used interchangeably with the term “subject” or “patient,” and includes humans, non human primates, dogs, cats, sheep, goats, cows, pigs, horses and rodents. In preferred embodiments, the individual is a human.

According to important aspects of the invention, methods for the diagnosis and treatment of individuals with gastrointestinal disease are provided. “Gastrointestinal disease,” as used herein, refers to duodenal ulcers and gastric atrophy. Gastrointestinal disease specifically does not include gastrinoma (Zollinger-Ellison Syndrome) or pernicious anemia.

“Duodenal ulcer” is characteristically a chronic and recurrent disease. These ulcers are usually deep and sharply demarcated. They penetrate through the mucosa and submucosa, often into the muscularies propria. The ulcer floor contains no intact epithelium and usually consists of a zone of eosinophilic necrosis resting on a base of granulation tissue surrounded by variable amounts of fibrosis. More than 95% of duodenal ulcers are carried in the first portion of the duodenum, with approximately 90% of those located within 3 cm of the junction of the pyloric duodenal mucosa. Duodenal ulcers are usually round and oval but they may be irregular or elliptic. They are usually less than 1 cm in diameter. Duodenal ulcers appear to be approximately as common in males as in females.

Gastric colonization with Helicobacter Pylori has been reported in 90-95% of patients with duodenal ulcer and in 60-70% of patients with gastric ulcer. Most patients with gastric colonization by H. pylori never develop ulceration and remain asymptomatic. H. pylori does not invade tissues. The organism resides in the mucus gel coating the epithelial cells with a minor proportion of H. pylori directly adherent to the epithelial cells.

“Gastric atrophy,” as used herein, refers to the final stage of chronic gastritis. Chronic gastritis initially involves the superficial and glandular areas of the gastric mucosa and progresses to glandular destruction, which may be followed by profound reduction in gland number (atrophy). With gastric atrophy there is a profound loss of the glandular structures which become separated widely by connective tissue with a greatly reduced or absent inflammatory infiltrate. The mucosa is thin, often revealing the prominence of its underlying vessels by endoscopic examination. As chronic gastritis progresses, there may be changes in the morphology of the gastric glandular elements. Intestinal metaplasia (gastric cancer) is the term used to describe the conversion of gastric glands to the appearance of small intestinal mucosal glands containing goblet cells. Intestinal metaplasia may be patchy or extensive in the gastric mucosa. With pseudopyloric gland metaplasia the glands of the body or the stomach assume the appearance of intrapyloric glands. Pseudopyloric metaplasia may occur with atrophic gastritis or gastric atrophy. Two major forms of chronic gastritis have been classified as types A and B based on their distributions in the gastric mucosa coupled with implications regarding their pathogenesis.

“Zollinger-Ellison Syndrome,” as used herein, refers to ulcer disease of the upper gastrointestinal tract marked with increases in gastric acid secretion from β-islet cell tumors of the pancreas. Diagnosis and treatment of these gastlin-containing tumors (also referred to as gastrinomas), is outside the scope of the present invention.

The current invention requires that the serum concentrations of gastrin, and in particular the concentrations of incompletely (nonamidated) and completely (amidated) processed forms of gastrin be determined in an individual with an elevated total gastrin level. As mentioned elsewhere, gastrin is a peptide hormone synthesized in the stomach that stimulates acid secretion through direct actions on parietal cells. Gastrin is initially synthesized as progastrin (nonamidated form), which is processed to glycine-extended gastrin (e.g., G-17-Gly, G-34-Gly, G-Gly: nonamidated forms), and then to amidated gastrin (e.g., G-17, G-34; amidated forms). A more detailed description of the gastrin molecules, including structures, is provided in “Gastrin”, by John H. Walsh and Dockray G J eds., 1994, New York: Raven Press, Ltd.

“Incompletely processed forms of gastrin,” as used herein, refer to nonamidated gastrins and include, progastrin (Gastrin 80), glycine-extended G-34 (G-34-gly), glycine-extended G17 (G-17-gly), Gastrin-71, Gastrin-66, and Gastrin-52. Preferred nonamidated gastrins are the progastrin, G-34-Gly, and G-17-Gly forms (G-34-Gly and G-17-Gly are often referred to throughout this application as G-Gly).

“Completely processed forms of gastrins,” as used herein, refer to amidated forms of gastrin and refer to G-17 and G-34 (the only currently known amidated gastrin forms).

According to the invention, the ratio is predictive of a risk for a gastrointestinal disorder in individuals with an elevated total gastrin level. “Total gastrin level,” as used herein, refers to a serum concentration of gastrin that is inclusive of all serum gastrin form concentrations (e.g., serum concentrations for the nonamidated and amidated forms). By “elevated,” as used herein, it is meant that the total gastrin concentration in an individual suspected of developing a gastrointestinal disorder is higher than the total gastrin concentration in a control individual. A control individual is a subject with no detectable disease, no detectable H. pylori infection, does not receive proton-pump inhibitor treatment or any other H. pylori anti-infective treatment, and has no prior history of a gastrointestinal disorder. A control individual does not have an elevated level of total gastrins.

Gastrin concentrations in an individual can be obtained by any art recognized method. Typically, the level is determined by measuring serum gastrin concentration in an individual. The level can be determined by ELISA, or immunoassays or other conventional techniques for determining the presence of the particular gastrin form (see also under the Examples, Wang, T C et al., J. Clin. Invest., 1996, 98:1918-1929, and Koh T J, et al., J. Clin. Invest., 1999, 103:1119-1126). Conventional methods include sending samples of an individual's body fluid to a commercial laboratory for measurement. The preferred body fluid is blood (serum). Gastrin measurements include both meal-stimulated and fasting gastrin measurements.

There presently are commercial sources which produce reagents for assays for the different gastrin forms. These reagents include, but are not limited to, polypeptides recognizing and binding to specific sequences (epitopes) in each gastrin. Such polypeptides include polyclonal and monoclonal antibodies, prepared according to conventional methodology (see, e.g., Clark, W. R. (1986) The Experimental Foundations of Modern Immunology, Wiley & Sons, Inc., New York; Roitt, I. (1991) Essential Immunology, 7th Ed., Blackwell Scientific Publications, Oxford). Once the body fluid concentrations for the nonamidated and amidated forms of gastrin are determined, a ratio of such concentrations can be easily obtained (see also under the Examples, Wang, T C et al., J. Clin. Invest., 1996, 98:1918-1929, and Koh T J, et al., J. Clin. Invest., 1999, 103:1119-1126).

The invention involves comparing the ratio of amidated to non-amidated gastrin obtained as described above, with a predetermined value. The predetermined value can take a variety of forms. It can be single cut-off value, such as a median or mean. It can be established based upon comparative groups, such as where the risk in one defined group is double the risk in another defined group. It can be a range, for example, where the tested population is divided equally (or unequally) into groups, such as a low-risk group, a medium-risk group and a high-risk group, or into quadrants or quantiles, the lowest being individuals with the lowest risk and the highest being individuals with the highest risk.

According to the invention, a predetermined value above about 1.1 indicates a higher than normal risk for developing duodenal ulcer disease. A predetermined value below about 0.9 indicates a higher than normal risk for developing gastric atrophy, with an increased likelihood of subsequently developing gastric cancer.

In an apparently healthy individual (a subject with no detectable disease, no detectable H. pylori infection, one that does not receive proton-pump inhibitor treatment or any other H. pylori anti-infective treatment, and one with no prior history of a gastrointestinal disorder) the ratio of nonamidated gastrins to amidated gastrins is about 1.0.

As discussed above, the invention provides methods for evaluating the likelihood that an individual will benefit from treatment with an agent for reducing risk of developing a gastrointestinal disorder. This method has important implications for patient treatment and also for clinical development of new therapeutics. Physicians select therapeutic regimens for patient treatment based upon the expected net benefit to the patient. The net benefit is derived from the risk to benefit ratio. The present invention permits selection of individuals who are more likely to benefit by intervention, thereby aiding the physician in selecting a therapeutic regimen. This might include using drugs with a higher risk profile where the likelihood of expected benefit has increased. Likewise, clinical investigators desire to select for clinical trials a population with a high likelihood of obtaining a net benefit. The present invention can help clinical investigators select such individuals. It is expected that clinical investigators now will use the present invention for determining entry criteria for clinical trials.

Accordingly, an individual with an elevated total gastrin level and a ratio of nonamidated gastrins to amidated gastrins between about 1.1 and about 1.75, or higher than 1.75, (i.e., above-normal, normal being a ratio of about 1.0), is highly likely to develop duodenal ulcer disease, and can therefore benefit from duodenal ulcer disease therapy. The individual may or may not have positive H. Pylori serology, and the individual may or may not be symptomatic for duodenal ulcer disease. In one embodiment, the individual has positive H. Pylori serology and is asymptomatic for duodenal ulcer disease. If the individual has dyspepsia and positive H. Pylori serology, further diagnostic studies (such as endoscopy) would become redundant, and duodenal ulcer disease therapy could be administered to the individual. Duodenal ulcer disease therapy involves administration of agents useful in treating duodenal ulcer disease. Currently, such agents include, but are not limited to: antacids such as aluminum hydroxide, magnesium hydroxide, magnesium trisilicate, calcium carbonate, and sodium bicarbonate; H-2 receptor antagonists such as Cimetidine, ranitidine, famotidine, and nizatidine; anticholinergic agents such as atropin and pirenzepine; coating agents such as colloidal bismuth; prostaglandins such as PGE₁ and PGE₂; proton-pump inhibitors, antibiotics, and/or combinations thereof.

Proton-pump inhibitors are agents that suppress gastric acid secretion by specific inhibition of the H⁺/K⁺ ATPase enzyme system at the secretory surface of the gastric parietal cell. Preferred proton-pump inhibitors include, but are not limited to, omeprazole (Prilosec), lansoprazole (Prevacid), rebeprazole (Acidphex), and pantoprazole.

Antibiotics include, but are not limited to, Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefinetazole; Cefinetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ometoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin, Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin.

According to another aspect of the invention, an individual with an elevated total gastrin level and a ratio of nonamidated gastrins to amidated gastrins below 0.9, preferably between about 0.9 and about 0.4 or lower (i.e., below-normal, normal being a ratio of about 1.0), is highly likely to develop gastric atrophy, and can therefore benefit from gastric atrophy therapy. The individual may or may not have positive H. Pylori serology, and the individual may or may not be symptomatic for gastric atrophy. Preferably, the individual is asymptomatic for gastric atrophy. In certain embodiments, the individual is a subject that receives proton-pump inhibitor treatment, and is preferably on chronic (long term) proton-pump inhibitor treatment (e.g., for gastroesophageal reflux disease/heartburn/dyspepsia). “Chronic proton-pump inhibitor treatment,” as used herein, refers to daily treatment of an individual with a proton-pump inhibitor for at least 3 months (90 days).

“Gastric atrophy therapy,” as used herein, refers to inhibition of parietal cell loss in an individual in need of such treatment (as determined according to the invention, see “gastrin ratios”) by administering an agent selected from the group consisting of a CCK-B/gastrin receptor antagonist, a proton-pump inhibitor, a nonamidated gastrin, or a combination thereof, in an amount effective to inhibit development of gastric atrophy in the individual. Any of the foregoing agents can be naturally isolated, or can be chemically synthesized. Gastrin molecules, for example, can be easily produced recombinantly.

In an important embodiment, gastric atrophy therapy involves the co-administration of a proton-pump inhibitor and a CCK-B/gastrin receptor antagonist. In one embodiment, the molar ratio of the proton-pump inhibitor and the CCK-B/gastrin receptor antagonist is 1:1. In further embodiments, the molar ratio of the proton-pump inhibitor and the CCK-B/gastrin receptor antagonist can be between about 2:1 and at least about 30:1. In yet further embodiments, the molar ratio of the proton-pump inhibitor and the CCK-B/gastrin receptor antagonist can be between about 3:1 and at least about 30:1. In particularly preferred embodiments gastric atrophy therapy involves the co-administration of a proton-pump inhibitor and a CCK-B/gastrin receptor antagonist, wherein the CCK-B/gastrin receptor antagonist only partially inhibits the CCK-B/gastrin receptor. “Partial inhibition,” as used herein, refers to “not completely inhibiting” gastrin secretion. As described above, individuals at risk of developing gastric atrophy and thought to benefit most firom the above-identified treatments are individuals on chronic proton-pump inhibitor treatment for gastroesophageal reflux disease.

“Cholecystoldnin-B(CCK-B)/gastrin receptor antagonists” are molecules that bind to the CCK-B/gastrin receptor and inhibit secretion of gastric acid via the CCK-B/gastrin receptor. Preferred CCK-B/gastrin receptor antagonists include, but are not limited to: L365,260; L740,093 (Merck); CI-988 (formerly PD-134,308; Parke-Davis); CAM-1028; CI-1015; PD135158; PD136450; PD140,376; GV150013X (Glaxo-Wellcome); LY288513 (Lilly); YM022 (Yamanouchi, Inc., Japan); YF476 (Ferring Research Institute/Yamanouchi); JB93182 (James Black Foundation); RP73870; RPR-101048; RB213; AG041R; DA-3934 (Daiichi Pharmaceutical); CR 2945 (see for example: Li Y, et al., American Journal of Physiology, 1999, 277(2 Pt 1):G469-77; Goddard A W, et al., Psychiatry Research, 1999, 85(3):225-40; Wiesenfeld-Hallin Z., et al., Behavioral & Brain Sciences, 1997, 20(3):420-5; discussion 435-513; Sandvik A K, and Dockray G J., European Journal of Pharmacology, 1999, 364(2-3):199-203; Kajbaf M, et al., Xenobiotica, 1998, 28(8):785-94; Luo B., et al., Brain Research, 1998, 796(1-2):27-37; Brenner L A, and Ritter R C., Physiology & Behavior 1998, 63(4):711-6; Trivedi B K., et al., Journal of Medicinal Chemistry, 1998, 41(1):38-45; Smadja C., et al., Psychopharmacology, 1997, 132(3):227-36; Rasmussen K., et al., Neuroscience Letters, 1997, 222(1):61-4; Semple G., et al., Journal of Medicinal Chemistry, 1997, 40(3):331-41; Lena I., et al., Journal of Neurochemistry, 1997, 68(1):162-8; Patel S., et al., Regulatory Peptides, 1996, 65(1):29-35; Hirst G C., et al., Journal of Medicinal Chemistry, 1996, 39(26):5236-45; Horwell D., et al., Immunopharmacology, 1996, 33(1-3):68-72; Helton D R., et al., Pharmacology, Biochemistry & Behavior, 1996, 53(3):493-502; Rasmussen K., et al., Neuroreport, 1996, 7(5):1050-2; Araldi G., et al., Farmaco, 1996, 51(7):471-6; Weng J H., et al., Bioorganic & Medicinal Chemistry, 1996, 4(4):563-73; Goudreau N., et al., Archiv der Pharmazie, 1996, 329(4):197-204)

As described elsewhere in the specification, preferred nonamidated gastrins are G-Gly (G-34-Gly and G-17-Gly) and progastrin.

As discussed above, the invention involves methods for treating individuals with abnormal gastrin ratios, to prevent gastrointestinal disorders. The agents are administered in amounts effective to lower the risk of the individual developing a gastrointestinal disorder.

An effective amount is a dosage of the agent sufficient to provide a medically desirable result. The effective amount will vary with the particular condition being treated, the age and physical condition of the subject being treated, the severity of the condition, the duration of the treatment, the nature of the concurrent therapy (if any), the specific route of administration and like factors within the knowledge and expertise of the health practitioner. It should be understood that the agents of the invention are used to reduce the risk of developing, or to treat gastrointestinal disorders, that is, they are used prophylactically in subjects at risk of developing a gastrointestinal disorder (asymptomatic), and acutely in subjects already symptomatic for the disorder. Thus, an effective amount is that amount which can lower the risk of, slow, reverse, or perhaps prevent altogether the development of a gastrointestinal disorder. It will be recognized that when the agent is used in acute circumstances, it is used to prevent one or more medically undesirable results. In the case of gastric atrophy, the agent can be used to limit parietal cell loss which develops as a result, for example, of chronic use of proton-pump inhibitors, and prevent the atrophy from progressing to gastric cancer.

Generally, doses of active compounds would be from about 0.01 mg/kg per day to 1000 mg/kg per day. It is expected that doses ranging from 50-500 mg/kg will be suitable, preferably orally and in one or several administrations per day. Lower doses will result from other forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of compounds.

For example, in the treatment of gastric atrophy, the usual dose given for the proton-pump inhibitor (e.g., omeprazole) is 20-40 mg/day, or 58-116 micromoles/day (the molecular weight of omeprazole is 345.42 grams/mole). This converts to 0.82 to 1.65 micromoles/kg/day. CCK-B antagonists, (e.g., YF476 M.W. 498.5 grams/mol, and YM022) are administered i.p. ranging from 50 nmol/kg/day to 25 micromol/kg/day (Ding et al, Pharmacology & Toxicology 1997;81:232-237). The ID₅₀ is about 50 nmol/kg/day which results in 50% inhibition of the CCK-B receptor effect. YM022 is reportedly about 5 times more potent than YF476 (Lindstrom et al, British Journal of Pharmcology, 1999, 127:530-536). Therefore, while the daily dose of the proton-pump inhibitor is around 1000-2000 nmol/kg/day, the dose of the CCK-B antagonist can be, for example, ˜50-100 nmol/kg/day, or 20 times less (than the proton-pump inhibitor), for ˜50% inhibition (partial inhibition of the CCK-B/gastrin receptor).

In another aspect, the invention provides pharmaceutical preparations comprising at least one agent selected from the group consisting of a CCK-B/gastrin receptor antagonist, a proton-pump inhibitor, and a nonamidated gastrin, in an effective amount to inhibit development of gastric atrophy (parietal cell loss) in an individual, and a pharmaceutically-acceptable carrier. Preferred nonamidated gastrins include G-Gly and progastrin.

When administered, the pharmaceutical preparations of the invention are applied in pharmaceutically-acceptable amounts and in pharmaceutically-acceptably compositions. Such preparations may routinely contain salts, buffering agents, preservatives, compatible carriers, and optionally other therapeutic agents. When used in medicine, the salts should be pharmaceutically acceptable, but non-pharmaceutically acceptable salts may conveniently be used to prepare pharmaceutically-acceptable salts thereof and are not excluded from the scope of the invention. Also, pharmaceutically-acceptable salts can be prepared as alkaline metal or alkaline earth salts, such as sodium, potassium or calcium salts.

Pharmaceutically-acceptable salts include, but are not limited to, those prepared from the following acids: hydrochloric, hydrobromic, sulfuric, nitric, phosphoric, maleic, acetic, salicylic, citric, formic, malonic, succinic, and the like. The pharmaceutical compositions may contain suitable buffering agents, including: acetic acid in a salt; citric acid in a salt; boric acid in a salt; and phosphoric acid in a salt.

The agents of the invention useful in treating gastrointestinal disorders may be combined, optionally, with a pharmaceutically-acceptable carrier. The term “pharmaceutically-acceptable carrier” as used herein means one or more compatible solid or liquid filler, diluents or encapsulating substances which are suitable for administration into an individual. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The pharmaceutical compositions also may contain, optionally, suitable preservatives, such as: benzalkonium chloride; chlorobutanol; parabens and thimerosal.

A variety of administration routes are available. The particular mode selected will depend, of course, upon the particular drug selected, the severity of the condition being treated and the dosage required for therapeutic efficacy. The methods of the invention, generally speaking, may be practiced using any mode of administration that is medically acceptable, meaning any mode that produces effective levels of the active compounds without causing clinically unacceptable adverse effects. Such modes of administration include oral, rectal, topical, nasal, interdermal, or parenteral routes. The term “parenteral” includes subcutaneous, intravenous, intramuscular, or infusion. Intravenous or intramuscular routes are not particularly suitable for long-term therapy and prophylaxis. They could, however, be preferred in emergency situations. Oral administration will be preferred for prophylactic treatment because of the convenience to the patient as well as the dosing schedule.

Compositions suitable for parenteral administration conveniently comprise a sterile aqueous preparation of the anti-inflammatory agent, which is preferably isotonic with the blood of the recipient. This aqueous preparation may be formulated according to known methods using suitable dispersing or wetting agents and suspending agents. The sterile injectable preparation also may be a sterile injectable solution or suspension in a non-toxic parenterally-acceptable diluent or solvent, for example, as a solution in 1,3-butane diol. Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or di-glycerides. In addition, fatty acids such as oleic acid may be used in the preparation of injectables. Carrier formulation suitable for oral, subcutaneous, intravenous, intramuscular, etc. administrations can be found in Remington's Pharmaceutical Sciences, Mack Publishing Co., Easton, Pa.

The pharmaceutical compositions may conveniently be presented in unit dosage form and may be prepared by any of the methods well-Inown in the art of pharmacy. All methods include the step of bringing the agent of the invention (e.g., a CCK-B/gastrin receptor antagonist, a proton-pump inhibitor, a nonamidated gastrin, anti-H.pylori), into association with a carrier which constitutes one or more accessory ingredients. In general, the compositions are prepared by uniformly and intimately bringing the agent of the invention into association with a liquid carrier, a finely divided solid carrier, or both, and then, if necessary, shaping the product.

Compositions suitable for oral administration may be presented as discrete units, such as capsules, tablets, lozenges, each containing a predetermined amount of the agent of the invention. Other compositions include suspensions in aqueous liquids or non-aqueous liquids such as a syrup, elixir or an emulsion.

Other delivery systems can include time-release, delayed release or sustained release delivery systems. Such systems can avoid repeated administrations of the anti-inflammatory agent, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the active compound is contained in a form within a matrix such as those described in U.S. Pat. Nos. 4,452,775, 4,675,189 and 5,736,152, and (b) diffusional systems in which an active component permeates at a controlled rate from a polymer such as described in U.S. Pat. Nos. 3,854,480, 5,133,974 and 5,407,686. In addition, pump-based hardware delivery systems can be used, some of which are adapted for implantation.

Use of a long-term sustained release implant may be particularly suitable for treatment of chronic conditions. Long-term release, are used herein, means that the implant is constructed and arranged to delivery therapeutic levels of the active ingredient for at least 30 days, and preferably 60 days. Long-term sustained release implants are well-known to those of ordinary skill in the art and include some of the release systems described above.

In another aspect, the invention provides novel diagnostic kits or assays which are specific for, and have appropriate sensitivity with respect to, the different gastrin forms, so that the nonamidated to amidated gastrin ratios could be determined. The preferred kits, therefore, would include, for example, agents that recognize and bind the various gastrin forms (e.g., anti-gastrin specific antibodies), as well as instructions or other printed material for characterizing risk based upon the outcome of the assay.

A kit embodying features of the present invention, generally designated by the numeral 11, is illustrated in FIG. 12. Kit 11 is comprised of the following major elements: packaging 15, a first agent of the invention 17 (e.g., a container with an anti-amidated gastrin specific antibody), a second agent of the invention 19 (e.g., a container with an antinonamidated gastrin specific antibody), and instructions 21 for utilizing such agents in diagnostic applications. Individuals skilled in the art can readily modify packaging 15 to suit individual needs.

The invention will be more fully understood by reference to the following examples. These examples, however, are merely intended to illustrate the embodiments of the invention and are not to be construed to limit the scope of the invention.

### EXAMPLES

### Example 1

Synergistic Interaction Between Hypergastrinemia and Helicobacter Pylori Infection in a Gastric Cancer Mouse Model.

The gastric hormone, gastrin, stimulates gastric acid secretion, and the growth of the acid secreting part of the stomach. In rats with experimental hypergastrinaemia (1, 2, 3), and in patients with gastrin-producing tumours of the Zollinger-Ellison syndrome (4, 5), there is an increase in the thickness of the acid-secreting mucosa, in the numbers of acid-secreting parietal cells and of histamine-secreting enterochromaffin-like (ECL) cells. However, mild or moderate hypergastrinaemia is also found in patients treated with acid-suppressing drugs, and in this case appears not to be associated with increased parietal cell numbers. Instead, most long term studies with proton pump inhibitors suggest that acid suppression that is associated with mild or moderate hypergastrinaemia occurs in the presence of parietal cell loss or gastric atrophy (6-9). The latter has often been linked to the confounding presence of Helicobacter pylori.

It is widely recognised that chronic H. pylori infection is associated with the development of chronic atrophic gastritis, a pathological condition characterized by decreased numbers of parietal cells and low levels of acid secretion. This has been linked to the presence of the micro-organism in the acid-secreting part of the stomach, and to the effect of both bacterial factors, and immunologic mechanisms on parietal cell loss. It is generally recognized that H. pylori infection results in a mild hypergastrinemia (1.5 to 2-fold elevated compared with uninfected subjects) that occurs early in the course of infection (10, 11), precedes the development of atrophic gastritis, and often resolves after eradication of the infection. While the role of hypergastrinermia in H.pylori in contributing to acid hypersecretion and duodenal ulcer disease has been well studied, a possible role in the progression to atrophic gastritis has not been directly addressed. The analysis is, in any case, complicated because once atrophic gastritis is established it would be expected to further contribute to increased plasma gastrin by reduced acid-inhibition of gastrin (G-)cell function.

I have examined the long term consequences of moderate hypergastrinaemia in a transgenic mouse model. In mice, unlike rats, but more like humans, it appears that hypergastrinaemia is associated with an early increase in parietal cell numbers, followed by a decrease which is exaggerated with Helicobacter infection. The present transgenic mouse model has the advantage of allowing studies of the temporal relationships between plasma gastrin and gastric epithelial cell function without the confounding variable of surgery or drug-induced hypochlorhydria commonly found in other experimental models.

Materials and Methods

Animals

The INS-GAS mice transgenic mice have previously been described (12, 13) and are specific pathogen-free. Animals were housed in a microisolator, solid-bottomed polycarbonate cages and fed a commercially prepared pelleted diet and given water ad libitum. For time course studies involving ECL and parietal cell number, serum gastrin levels, and maximal acid secretion, a minimum of four mice were used for each time point. For long term studies, eight INS-GAS mice and eighteen wild type FVB/N mice assayed at 20 months of age. For infection studies, six wild type FVB/N (Taconic Farms) and eight INS-GAS mice at four weeks of age were inoculated with H. felis (ATCC 49179) as previously described (14). Infection status was confirmed by ELISA assays measuring IgG antibody to H. felis at 4 weeks and 6 months post-inoculation, and by quantitative urease assays of gastric tissue at necropsy (15). All experiments were approved by the Subcommittee on Research Animal Care at Massachusetts General Hospital and the Committee on Animal Care at MIT.

Acid Measurements

After food deprivation (free access to water) for 24 hours, the mice were anesthetized by inhalation of methoxyflurane (Pitman-Moore, Inc., Mundelein Ill.). The abdomen was opened by midline incision and the gastric content was collected by rinsing the stomach with 1 ml saline (37° C.) through a canula placed in the pylorus. The pylorus was then ligated by applying silk suture material firmly around the junction between the pylorus and the duodenum, followed by abdominal closure. Mice recovered from anesthesia 5 min after removal from methoxyflurane exposure and completion of surgery. Acid secretion was then measured using the pyloric ligation model similar to that prevously described (16). After 4 hours of pyloric ligation, the entire volume of gastric juice was then collected via a stomach tube under similar anesthesia. The acidity of the gastric contents was measured by titration with 0.02 N NaOH and expressed as H+ nEq. The mice were subsequently euthanized by Co2 asphyxiation.

Histology

At necropsy, the stomach was opened and ingesta removed. The animals were examined for gross changes, and the tissue specimens collected consisted of the gastric mucosa and wall beginning at the gastroesophageal junction and ending just beyond the gastroduodenal junction. Stomach tissue specimens were fixed in neutral buffered 10% formalin, processed by standard methods, embedded in paraffin, sectioned at 5 μm, and stained with H&E (for histopathologic assessment) and Alcian blue at pH 2.5/PAS (for acid sailomucin detection). Additional sections were cut for immunocytochemistry, and separate samples processed for Western blot or RIA. Samples were also fixed and embedded in a mixture of Epon 812 and Araldite Epoxy Resin 6005, and processed for electron microscopy as previously described (14). Semithin sections (0.5 μm thick) were stained with 1% toluidine blue in 1% borate for light microscopy. Adjacent thin sections (0.08-0.1 μm thick) were stained with uranyl acetate and lead citrate and examined with a JEOL 1200 EX electron microscope.

Immunocytochemistiy and Quantitation of Parietal and ECL Cells:

Immunocytochemistry was performed as previously described (14). Parietal cells were detected using the rabbit polyclonal anti H+, K+-ATPase (Ab 12.18, 1:1000, gift of Dr. Adam Smolka). Spasmolytic polypeptide (SP) cells were detected using the murine monoclonal antibody against SP (1:20, gift of Nicholas Wright). BrdU was performed as previous reported (17). Staining HB-EGF (M-18, Santa Cruz Biotechnoglogy, Inc., Santa Cruz, Calif.) and for TGFα (rabbit anti-mouse TGFα, East Acres Biological, Southbridge, Mass.) were performed using commercially available antiserum. Sections were counterstained with hematoxylin alone. Parietal cells were counted as previously reported (18). Five high power fields in each H&E stained section were examined, beginning at the junction between forestomach and fundus, with two well oriented gastric glands counted per field for a total of ten glands per mouse.

For histamine staining of ECL cells, small specimens from the oxyntic part of the stomach were fixed in 4%1-ethyl-3(3-dimethyl-aminopropyl)-carbodiimide hydrochloride (Sigma, St. Louis, USA) for 8-12 h at 4 C., and then rinsed with 20% sucrose in 0.1M phosphate buffer (pH 7.4). The specimens were frozen at −80° C. and cut in a cryostat (Bright, Huntington, England) at 10 μm thickness and mounted on gelatin-coated slides. The primary antiserum was raised in rabbit (# 8431) and used in a final dilution of 1:1000 in phosphate-buffered saline (pH 7.4), containing 0.25% Triton X-100 (PBS-T) (19). The sections were incubated with the antibody over night at 4° C. After washing twice (10 min) with PBS, the sections were incubated for 1 h at room temperature with fluorescein-conjugated goat antiserum against rabbit immunoglobulins (IgG) (# F0205, Dalco, Glostrup, Denmmark) diluted at 1:40 in PBS-T. After washing twice (10 min) with PBS, the sections were mounted in PBS: glycerol (1:4) and examined in light microscope (Olympus BX50, Japan). The positive cells were counted and expressed as number per mm of the mucosa.

Western Blots

Frozen stomach tissues were allowed to thaw on ice in the presence of 0.5 ml to 1.0 ml of ice-cold lysis buffer containing 10 mM Hepes pH 7.4, 30 mM NaCl, 0.3 mM MgCl2, 0.2 mM EDTA, 0.2% Triton X-100, 2% glycerol, 1 mM sodium orthovanadate, 1 mM sodium fluoride, and EDTA-free complete protease inhibitor (Boehringer Mannheim). Tissues were sonicated for 2×20 sec with probe type sonicator and centrifuged at 10,000 rpm for 20 min at 4° C. Supernatants were collected and protein concentrations determined. 100 mg of lysates were electrophoresed on 12% (for CCK-B) or 16% (for HB-EGF) SDS polyacrylamide gel and transferred to Hybond-ECL nitrocellulose membrane (Amersham). HB-EGF and CCK-B receptor proteins were detected by enhanced chemiluminescence method according to the manufacturer's directions, using 1:1000 dilution of primary antibody and 1:10,000 of the secondary antibody. Western blotting for the CCK-B receptor was carried out using the rabbit antiserum to human CCK-B (#95161, courtesy of John H. Walsh and Helen C. Wong, CURE/UCLA). The anti-HB-EGF (M-18) antibody and the corresponding secondary antibody (anti-goat IgG-HRP, preabsorbed) were purchased from Santa Cruz Biotechnology, Inc (Santa Cruz, Calif.).

Gastrin and TGFα Measurements

Human amidated gastrin-17 was measured using the L6 antibody, and total amidated gastins (human and mouse) were measured using the L2 antibody in radioimmunoassays as described (12). TGF-α present in gastric tissue was also measured by RIA as previously reported (20). Statistics (Students t-test or Fisher test) were calculated using Microsoft Excel software.

Results

Hypergastrinemic (INS-GAS) Mice Show Initial Increases Followed by Sustained Decreases in Acid Secretion and Parietal Cell Number

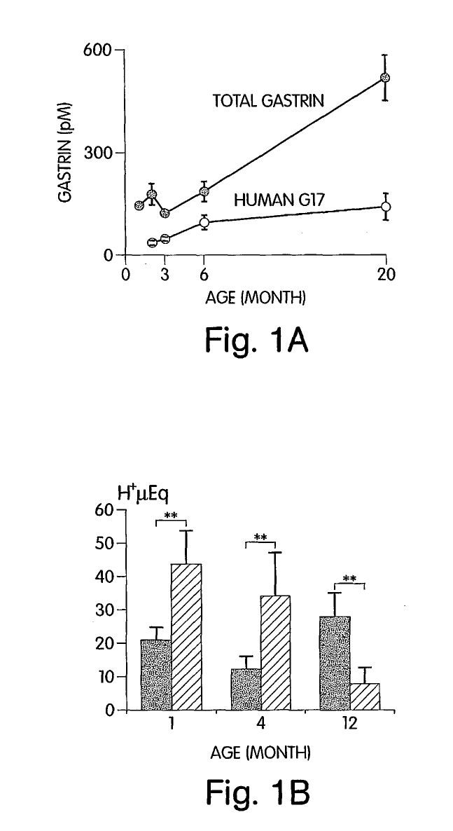

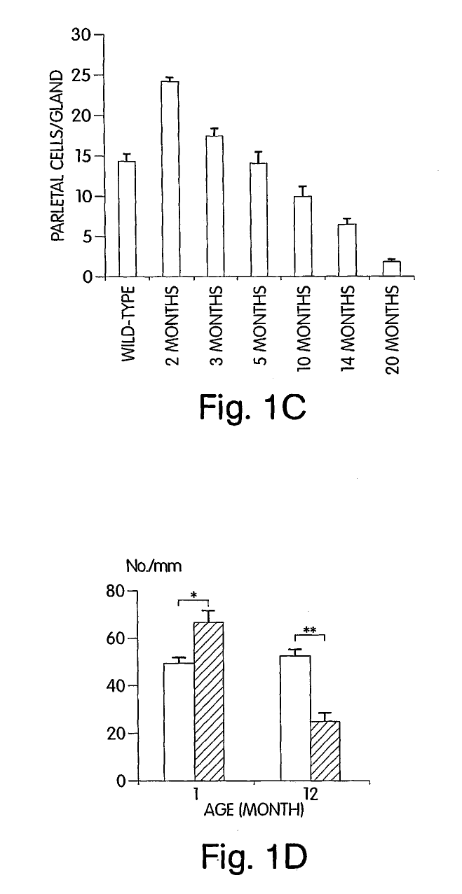

To investigate the long-term effects of hypergastrinemia on the gastric mucosa, we studied insulin-gastrin (INS-GAS) transgenic mice which contain a chimeric transgene in which the human gastrin gene is transcribed from the rat insulin I promoter (12). These mice express human heptadecapeptide gastrin (G-17) in the pancreastic β cells which is then secreted into the circulation. We analyzed the effects of increased gastrin secretion over time in these mice, and found that the changes could be divided into two distinct phases: an early phase (1-4 months) and a later phase (5 months and older). During the early phase, gastrin levels were modestly increased (149-180 pM), and approximately twofold elevated compared to wild type FVB/N mice (73 pM, p<0.05, t test) (13). Slightly more than 50% of the serum gastrin (and all of the increase) in these young INS-GAS mice was human gastrin resulting from pancreatic secretion, and was thus derived from the transgene, as demonstrated with a human G-17 specific antibody (FIG. 1A). In contrast to the elevated plasma amidated gastrin levels, the levels of glycine-extended gastrin in these mice were all <20 pM. The consequences of these mild increases in gastrin were examined in INS-GAS mice, relative to wild type controls, using the pyloric ligation model which can be used to assess maximal acid output. During the early gastrin phase (1 and 4 months), INS-GAS mice showed a 2-3-fold increase in stimulated acid secretion (FIG. 1B), consistent with the early elevations in serum gastrin. There was no significant change in the volume of gastric juice secreted (not shown). The increase in stimulated acid secretion was also consistent with early (2 and 3 month) increases in the number of parietal (FIG. 1C). Enterochromaffin-like (ECL) cell number measured at 1 month of age also showed a small but significant increase (FIG. 1D).

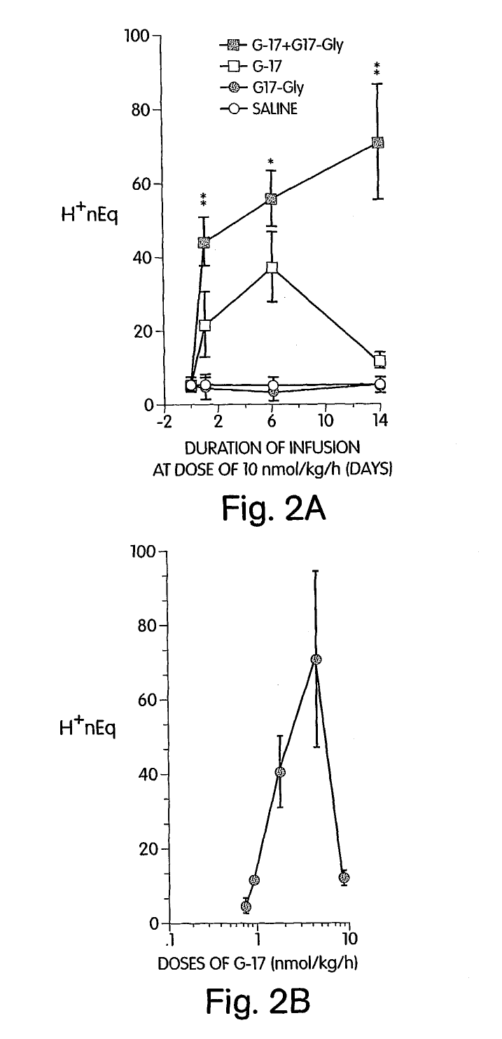

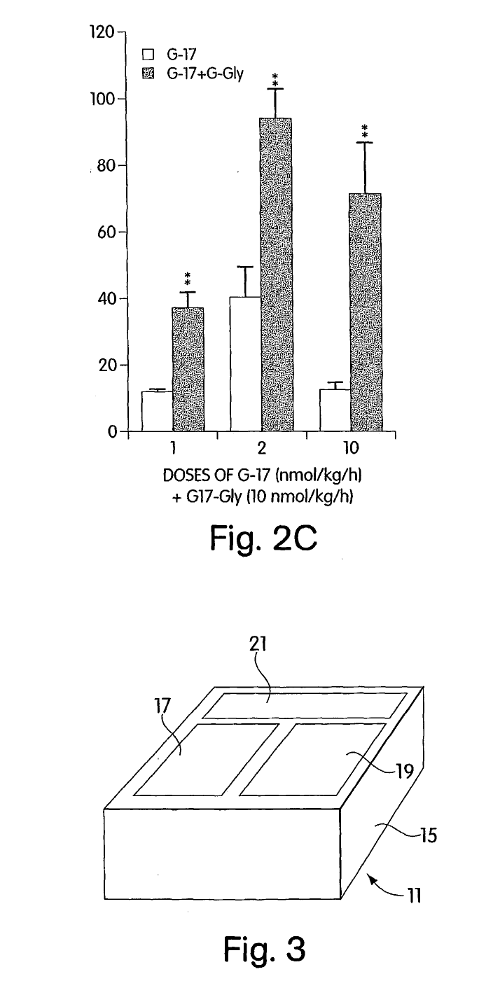

During the late gastrin phase (5 months and older), the INS-GAS mice showed a gradual change in gastric function with a marked decline in the level of stimulated acid secretion. At 12 months of age, the level of acid secretion was significantly reduced compared to that of wild type mice (FIG. 1B). At 20 months of age, the INS-GAS mice secreted essentially no acid. These decreases in acid production were mirrored by decreases in parietal and ECL cell numbers; parietal cells numbers declined to <68% of normal by 10 months, and to <40% of normal by 14 months of age (FIG. 1C). Although previous reports by our group raised the possibility of increased parietal cell number in 10-12 month old INS-GAS mice (13), strict quantitation showed an unambiguous decrease in parietal cells at this time point. By 20 months, only a small number of parietal cells were still present; these were localized mainly in the base of the gastric glands, and parietal cells were no longer detectable in the gastric pit region. ECL cell numbers fell well below normal by 12 months of age (FIG. 1D), and also showed an altered distribution with increasing numbers found in the upper half of the gastric glands. The decline in parietal and ECL cells at 20 months was reflected in the decreased expression of the CCK-B/gastrin receptor as shown by western blots (FIG. 2A). Serum amidated gastrin levels rose gradually beginning at 6 months, with levels peaking at 550 pM by 20 months of age (FIG. 1A). Most of the increase in serum gastrin resulted from increased secretion of endogenous mouse amidated gastrin, with lesser changes in the level of transgene-derived human gastrin (human G17), which rose from 49 pM at 3 months to 142 pM at 20 months (FIG. 1A).

INS-GAS Mice Show Increased Expression of TGFα and HB-EGF

Prolonged hypergastrinemia led to the development of foveolar hyperplasia in the INS-GAS mice. Concurrent with the decline in parietal cell number and the size of the glandular compartment, there was an expansion of surface mucous cells (gastric pit region) leading to an overall increase in mucosal thickness. Over time, the histology of the INS-GAS stomach resembled Menetrier's disease, a disorder in human patients associated with marked foveolar hyperplasia and thickened gastric folds. Since Menetrier's disease has been reported in association with increased expression of TGFα (21), we examined expression of TGFα as well as HB-EGF, a member of the EGF/TGFα family that has recently been shown to be expressed in parietal cells and regulated by gastrin (22, 23). Increased expression of TGFα could be detected by RIA of fundic mucosal extracts, with significant differences measured at 9 and 10 months of age. At 20 months of age, the differences were not significant but focal areas of markedly increased expression could be detected by inmunocytochemistry. Western blots showed moderate increases in HB-EGF expression at 2 and 3 months, and markedly increased expression at 20 months. Immunocytochemistry revealed that in 20 month old INS-GAS mice HB-EGF staining was more intense in parietal cells and was also present in foveolar cells. The increased expression of these growth factors in the INS-GAS mice was associated with increased proliferative indices, and BrdU staining showed broadening of the proliferative zone now located at the base of the gastric glands (13).

INS-GAS Mice Develop Metaplasia, Dysplasia and Invasive Gastric Carcinoma

INS-GAS mice at 20 months of age showed marked gastric hypertrophy. Grossly, the hypertrophy involved only the fundus of the stomach and resulted in thickened gastric folds, while the gastric antrum appeared uninvolved. The hypertrophy of the oxyntic glands was due primarily to marked foveolar hyperplasia, which was associated with only minimal glandular tissue still present in the older mice. Alcian Blue staining at pH 2.5, which is specific for acid sialomucins normally absent from the fundic mucosa, showed positive staining and thus evidence of intestinal metaplasia. In addition, there was expansion of an aberrant spasmolytic peptide (SP or TFF2)-positive lineage at the base of the gastric glands. This SP (+) lineage is associated with parietal cell loss in inbred strains of mice that develop preneoplasic changes in response to Helicobacter felis infection (14), and increased expression of TFF2 has been observed in human gastric cancer as well (24). All mice >20 months showed clear evidence of intramucosal dysplasia, and most older mice were found to exhibit intramucosal carcinomas. The majority of older mice (6 out of 8) followed for 20 months of age developed gastric carcinomas that invaded the submucosa, muscularis, and vasculature. The diagnosis of gastric cancer was confirmed by electron microscopy studies demonstrating poorly differentiated carcinoma cells with prominent nucleoli, increased nuclear-to-cytoplasmic ratios and decreased intercellular contact. There was no evidence of metastases to lymph nodes or other tissues. Eighteen 20 month old wild type FVB/N mice showed no evidence of metaplasia, dysplasia, or carcinoma.

Helicobacter Infection in INS-GAS Mice Accelerates Progression to Gastric Carcinoma

Since certain inbred strains of mice develop parietal cell loss and atrophy following Helicobacter felis infection (14,25), we examined the possible role of hypergastrinemia in accelerating the development of gastric atrophy and cancer in response to Helicobacter infection. INS-GAS mice and wild type mice in the same genetic background (FVB/N) were infected with H. felis at 4 weeks of age, and monitored for up to seven months post-inoculation. Uninfected INS-GAS and FVB/N mice were utilized as control animals in these studies.

In our initial study, mice were followed for up to 5-6 months post-inoculation. The serum H. felis-antibody responses in both sets of infected mice were equivalent at 4 weeks and 5-6 months post-inoculation. At four weeks post-inoculation, H. felis-infected INS-GAS mice had antral urease scores (63.2±17.1 OD/mg tissue×1000) that were similar to those of infected wild type mice (59.1±8.1, p=0.14), but fundic urease scores (39.3±6.4) that were higher than those of the infected wild type controls (17.1±5.8, p<0.05), consistent with increased findic colonization. At 5-6 months post-inoculation, both the uninfected INS-GAS mice and the infected FVB/N wild type mice showed mild parietal cell loss and foveolar hyperplasia in comparison to uninfected FVB/N mice. However, H. felis infected INS-GAS mice exhibited profound alterations in gastric epithelial architecture with severe foveolar hyperplasia, atrophy, metaplasia, and clear evidence of dysplasia. In this initial study, at 5-6 months post-inoculation, 1/4 infected INS-GAS mice showed intramucosal and submucosal carcinomas; no cancers were observed in the other three groups of mice (4 mice/group).

A larger series of mice (8-15 mice/group) with or without H. felis infection was then studied for up to 6-7 months post-inoculation. This study included fifteen (15) INS-GAS mice and fourteen (14) FVB/N mice infected with H. felis; eight (8) INS-GAS and eight (8) FVB/N mice enrolled in the study remained uninfected. Infection was confirmed in both sets of mice at all time points through H. felis-specific serum ELISA assays. At approximately 4 months post-inoculation, the infected INS-GAS mice began to show a gradual decline in health, while the other three groups remained robust. Two mice infected INS-GAS mice had to be sacrificed prematurely (one at 4 months, one at 5 months) for health reasons; both showed evidence of intramucosal carcinomas, and submucosal and intravascular (invasive) carcinoma was also present in the mouse sacrificed at 5 months post-inoculation. At six months post-infection, three additional infected INS-GAS mice showed signs of deteriorating health, and thus the study as a whole was terminated between 6 and 7 months post-inoculation.

At 6-7 months post inoculation, plasma gastrin levels in the infected (295 pM) and uninfected (192 pM) INS-GAS mice were significantly elevated compared to levels in uninfected FVB/N mice (43 pM, p<0.05). Interestingly, plasma gastrin levels were not elevated in the infected FVB/N mice (41 pM). Quantitative urease assays indicated a significant decrease at 6-7 months post-inoculation in the level of colonization in the infected INS-GAS mice. Histopathologic analysis showed all infected INS-GAS mice had severe atrophy. The degree of atrophy was quantitated through parietal cell counts; the number of parietal cells/gland was decreased by 27% in the infected FVB/N mice, by 40% in the uninfected INS-GAS mice, and by 67% in the infected INS-GAS mice.

In addition, infection of INS-GAS mice with H. felis resulted in profound changes in mucosal architecture. All of the infected mice showed evidence of multifocal dysplasia, characterized by epithelial atypia, hyperchromicity, and epithelial disorganization and stratification. The majority of infected INS-GAS mice showed evidence of neoplastic progression, as manifested by intramucosal carcinomas. Half of infected mice showed carcincinoma invasive into the submucosa and vasculature. Neoplastic foci were comprised of well-differentiated, invasive neoplastic epithelial population, which arose from proliferative epithelium with a metaplastic mucin phenotype (i.e., acid sialomucins). The intramucosal carcinomas most often had well-differentiated tubulo-acinar or micro-acinar architecture, resulting in irregular, cystic, or minute glandular tubules or acini. The epithelium ranged from a primitive, hyperchroniic columnar epithelium to a secretory epithelium with clear apical mucin. In some cases, intraepithelial formation of mucous acini was observed. Invasion through the muscularis mucosae into the submucosa was often associated with invasion into veins. In some cases, thrombosis was observed in tandem with vascular invasion. Neoplastic foci invading into the submucosa and vasculature, were often enclosed by fibrous capsules. Overall, intramucosal carcinoma was present in 11/13 (85%, p<0.01) mice, invasive submucosal carcinoma in 7 of 13 (54%, p<0.01) mice, and intravascular invasion in 6 of 13 (46%, p<0.05) mice. An intramucosal carcinoma was observed in this study in 1/8 uninfected INS-GAS mouse, but not found in any of the other two groups.

### Example 2

Glycine-extended Gastrin Synergizes with Gastrin-17 to Stimulate Acid Secretion in Gastrin Deficient Mice.

The role of gastrin in the regulation of acid secretion and growth of the stomach is well recognized. Recent work has provided support for the view that the action of gastrin as a secretogogue is due mainly to stimulation of histamine release from the enterochromaffin-like (ECL) cells of the oxyntic mucosa (47, 48). The relative importance of gastrin's direct effects on parietal cells are less clear cut, although in the past it has been argued that gastrin stimulates parietal cells directly to secrete acid, and also increases sensitivity, or responsiveness, to histamine and acetylcholine. It is generally accepted that CCK-B/gastrin receptors are present on parietal cells and studies in a number of animal models have shown that hypergastrinemic states lead in the short term to significant increases in parietal cell number (49, 50).

Recent studies in gastrin deficient mice have supported a key role for gastrin in both acid secretion and gastric epithelial cell differentiation. Gastrin deficient mice have been shown by several groups to have a profound impairment in gastric acid secretion, beyond that predicted from the modest reduction (˜30%) in parietal cell number alone (ECL number was unchanged), suggesting a primary defect in parietal cell maturation (51, 52). In addition, short term infusions of amidated gastrin for 6 days resulted in only partial (˜30%) restoration of basal acid secretion in gastrin deficient mice (52).

Most of the original studies which examined the role of gastrin as a secretogogue focused solely on the amidated gastrin peptides G-17 and G-34. However, it is now clear that the less processed forms of gastrin (progastrin and glycine-extended gastrin) have biological activity in their own right.

It has been reported that both progastrin and glycine-extended gastrins (G-Gly) have trophic effects on gastrointestinal tissue in both in vitro and in vivo model (53,54). Several studies have examined the possible effects of G-Gly as a direct stimulant of acid secretion. Studies in normal human subjects demonstrated no effect on gastric acid secretion by G-Gly (55,56), while studies in rats suggest that G-Gly might potentiate gastrin-stimulated acid secretion (57,58). However, all of these earlier studies were carried out over relatively short time periods; for example, the human studies represented infusions of less than 8 hours. The interpretation of these studies is further complicated by the fact that they were performed of background concentrations of circulating gastrin peptides. In the present study, we used gastrin deficient mice that lack all progastrin-derived peptides to examine the role of amidated and and G-Gly gastrin alone, and in combination, on the regulation of acid secretion. The acid secretion was evoked by pyloric ligation which is known to be mediated via vagal excitation (60). These studies show a key role for glycine-extended gastrin in the long term maintenance of normal acid secretion.

Materials and Methods

Animals

Gastrin deficient mice were generated through targeted gene disruption as previously described (51). Animals were operated and sacrificed under anesthesia with Metofane. The study was approved by the MGH Animal Research Committee (Boston, Mass., USA).

Experimental Design

Sixty gastrin deficient mice were randomized to receive an infusion of saline, or 1, 6 or 14 days of either gastrin-17 (G-17), G17-Gly, or a combination of the two at rates of 10 nmol/kg/hr via subcutaneous osmotic minipumps (ALZA Corporation, Palo Alto, Calif.). The minipumps were activated by incubation in 0.9% NaCl at room temperature overnight and then implanted under the skin in the neck (under anesthesia). The mice were fasted 24 hours prior to sacrifice. The mice were given BrdU (50 mg/kg i.p.) 1 hour prior to sacrifice.

Gastric Acid Secretion

After food deprivation for 24 hours, the mice were anesthetized with avertin, the abdominal cavity was incised, and each stomach was rinsed with 1 ml saline through a canula placed in the pylorus. The pylorus was then ligated and the gastric juice was collected 4 hours later (61). The acidity of the gastric contents was measured by titration with 0.02 N NaOH and expressed as H⁺ nEq.

Serum Gastrin Concentation

Blood from each mouse was drawn from the abdominal aorta at sacrifice. Gastrin concentrations were determined by radioimmunoassay using antibodies to the COOH-terminus of glycine-extended gastrin and amidated gastrin (antibodies 109-21 and L2 respectively) as previously described (54).

Immunohistochemistry

At the time of sacrifice, the stomach was divided into thirds longitudinally. One section was fixed in Carnoy's fixative overnight, and then embedded in paraffin. Routine H&E staining was performed. Parietal cells were stained using an antibody to the β subunit of H+/K+-ATPase MA3-923 (Affinity Bioreagents, Golden, Colo.) at a final dilution of 1:100, as previously described (62). Ten well oriented fundic glands were examined for parietal cell number, with the result expressed as number of parietal cells per gland (51).

Immunohistochemical detection of BrdU incorporation was performed using the avidin-biotin monoclonal antibody technique as previously described (51). Ten well oriented oxyntic glands or colonic crypts were counted per slide. Of the ten colonic crypts, 4 were obtained from the rectum, 3 from the descending colon, and 3 from the ascending colon. The BrdU labeling index was calculated by counting the number of BrdU-positive cells per gland/crypt and expressing the result as a percentage of the total number of cells per gland/crypt.

Small tissue specimens (2×4 mm) were taken from the greater curvature of the oxyntic mucosa. They were fixed either in 4% 1-Ethyl-3(3-dimethylaminopropyl)-carbodiimide hydrochloride (Sigma, St. Louis, Mo.) for histamine staining, or in 4% paraformaldehyde for VMAT-2 staining, for 8-12 h at 4° C., then rinsed with 20% sucrose in 0.1 M phosphate-buffered saline (pH 7.4). Fresh frozen tissue sections were cut in a cryostat (Bright, Huntington, England) at 10 μm thickness. The sections were thawed onto gelatin-coated glass slides. After washing twice for 10 min with phosphate-buffered saline (pH 7.4), containing 0.25% Triton X-100 (PBS-T), the sections were incubated with either histamine antibody 8431 rabbit (Eurodiagnostic, Mahnö, Sweden) at a final dilution of 1:200 or VMAT-2 antibody B-GP 280-1 (Alpha Diagnostic Int., San Antonio, Tex.) at a dilution of 1:2000 overnight at 4° C. After washing twice with PBS-T (10 min each time), the sections were incubated for 1 h at room temperature with FITC-conjugated goat anti-rabbit IgG (Dako, Glostrup, Denmark) for histamine at a 1:40 dilution or donkey anti-guinea pig IgG (Jacksson InmunoResearch, Göteborg, Sweden) for VMAT-2 at a 1:100 dilution. After washing with PBS twice (10 min), the sections were mounted in PBS: glycerol (1:4) and examined in a light microscope (Olympus BX50, Japan). The number of immunostained cells were counted and exressed as number per gland of mucosa.

Electron Microscopy

Small tissue specimens (<1 mm³) were taken from the oxyntic gland area and immediately immersed in a mixture of glutaraldehyde from the oxyntic gland area and immediately immersed in a mixture of 1% glutaraldehyde, 3% formaldehyde, 0.075M sodium phoshpate buffer, pH 7.2, for 6 hours. The specimens were post-fixed in 1% osmium tetroxide for 1 hour, dehydrated in graded acetone solutions and embedded in Epon 812. Ultrathin sections (60-80 nm) were cut on a LKB MK III Ultratome, contrasted with uranyl acetate and lead citrate and examined in a Philips CM10 (Philips,) transmission electron microscope (63). Parietal cells were identified by the presence of secretory canuliculi and/or tubulovesicles, and ECL cells were identified by the presence of characteristic granules and vesicles in the cytoplasm. Point counting methods were used to analyze the voulume density of the various organelles as previously described (64,65). The cells were photographed (nucleated profiles only) at 6,000× magnification; the prints were then enlarged to 20,000× for planimetric analysis. Activated parietal cells were defined as cells having secretory canuliculi in their cytoplasm. The percentage of activated parietal cells was determined by analyzing the electron micrographs of 100 parietal cells per group. Vacuolar canaliculi were defined as canuliculi containing lysosomal bodies and/or vacuoles in their lumen. Lipofuscin bodies were identified by their high electron density (osmiophilia) in the cytoplasm.

Parietal Cell and ECL Cell Counts

Parietal and ECL cells were stained immunohistochemically as above. At least ten well oriented glands were then assessed for the number of positively stained cells per gland.

Statistical Analysis

The results were expressed as means±SEM. ANOVA test for random differences was applied. P<0.01 was considered statistically significant.

Results

Serum Amidated Gastrin and G17-gly Concentrations

Infusion of G-17 resulted in elevated levels of G-17 (1742.9±607.2 pM) compared to saline infused mice, whose levels were below the limits of detection (<20 pM), when measured one day prior to sacrifice. Levels of glycine-extended gastrin were below the limits of detection (<20 pM) in both groups. Infusion of G17-Gly resulted in elevated serum levels of G17-Gly (1525.0±291.5 pM) when compared to saline infused mice (<20 pM), with undetectable amounts of G-17 (<20 pM).

G17-Gly Enhances G-17-induced Acid Secretion

Infusion of G-17 resulted in a steady increase in acid levels after one day (21.8±8.8 nEq) and six days (37.4±9.4 nEq) (FIG. 2) when compared to saline infusion (5.4±2.0 nEq). However, after 14 days of G-17 infusion, the acid output decreased to 12.0±2.2 nEq. Infusion of G17-Gly alone had little impact on acid. However, infusion of G17-Gly in addition to G-17 resulted in a marked elevation of acid output at day 1 (44.2±6.4, p<0.01), day 6 (56.0±7.6, p<0.05), and day 14 (71.4±15.8, p<0.01) (FIG. 2) which was statistically significant when compared to mice receiving G-17 alone.

G17-Gly Had No Effect on G-17 Induced Gastric Proliferation

Infusion of G-17 resulted in a significantly increased proliferation rate of the gastric fundic mucosa at one day when compared to infusion of saline, as measured by BrdU incorporation. G17-Gly infusion had no effect on proliferation rates when compared to saline infusion, and also had no effect when given in combination with G-17 when compared to G-17 infusion alone.

G17-Gly Had No Effect on G-17 Induced Parietal Cell Hyperplasia, or on ECL Cell Number

Infusion of G-17 resulted in a gradual increase in the number of parietal cells compared to infusion of saline, with a statistically significant 65% (p<0.01) increase by day 14. G17-Gly had no effect on parietal cell number when compared to saline, and had no additive effect when given in combination with G-17 compared to G-17 alone. Interestingly, neither G-17, G17-Gly, or the combination influenced ECL cell number as indicated by histamine (FIG. 9a) or VMAT-2 staining at any time point examined.

G17-Gly Had No Effect on H+/K+-ATPase Expression.

It has been previously reported that G17-Gly increases H+/K+-ATPase mRNA abundance (66). In order to determine whether the increase in acid secretion seen with gastrin reinfusion was secondary to increased expression of H+/K+-ATPase, immunohistochemical analysis was performed. As previously noted, infusion of G-17 alone resulted in a significant increase in parietal cell number when compared to saline alone, and the addition of G-17-Gly had no effect on parietal cell number. However, while an increased number of parietal cells was evident with immunostaining for H+/K+-ATPase in G-17 treated mice, there was no discemable effect on the intensity of staining after infusion of G-17+G-Gly when compared to infusion of saline alone. Infusion of G-17 or G-Gly alone also had no effect on H+/K+-ATPase expression.

G17-Gly Enhances G-17 Induced Activation of Parietal Cells.

Electron microscopy allowed the identification of resting parietal cells versus activated parietal cells. After 14 days of infusion, there was no significant increase in the number of activated parietal cells in mice receiving G17-Gly (26%) or G-17 (27%) when compared to mice receiving saline alone (27%). However, mice receiving a combination of G17-Gly and G-17 had a significant increase in the number of activated parietal cells (86%) compared to all other groups. Interestingly, mice receiving a 14 day infusion of G-17 alone had a significant increase in the presence of vacuolar canaliculi (7.50±2.40% vs. 0.38±0.38%, p<0.01) and lipofuscin bodies (1.88±0.27% vs. 0.26±0.10%, p<0.01) when compared to saline infusion (expressed as a percentage of cell volume). The addition of G17-Gly to G-17 prevented the increase of both vacuolar caniliculi and lipofuscin bodies seen with infusion of G17 alone. Infusion of G-Gly alone had no effect on either of these parameters when compared to saline infusion.

### DETAILED DESCRIPTION OF THE DRAWINGS

FIG. 1. INS-GAS mice demonstrate loss of initial gastric acid hypersecretion and increasing serum gastrin levels over time. (A) Serum gastrin levels in INS-GAS mice over time. Both total amidated gastrin (closed circles) and human amidated gastrin (open circles, human G-17, derived from the transgene) are shown. (B) Gastric acid output (H μEq⁺) in INS-GAS mice and wild-type (WT) control mice at 1, 4, and 12 months. Control mice revealed no significant change in either parameter with age. Each data point represents measurements from 5-6 mice. (**) indicates significant difference (p<0.05, t-test) compared to WT mice of the same age. (C) Parietal cell counts over time. There was no change in parietal cell numbers for wild type mice at 2 and 20 months, and data from these two time points has been combined into a single data point for comparison. The decrease in parietal cell number compared to controls was statistically significant at 2 and 3 months (increased) and at 10, 14, and 20 months (decreased) (p<0.05, t-test). (D) ECL cell number in wild type and INS-GAS mice at 1 and 12 months of age. (*) indicates signficant difference (p<0.05) compared to wild type (WT), while (**) indicates significant difference compared to both WT and 1 month old INS-GAS.

FIG. 2. G17-Gly augments G-17 induced acid secretion in gastrin deficient mice. Gastrin deficient mice received infusions of saline, G17-Gly, G-17, or G-17+G17-Gly for 1, 6 or 14 days. Acid secretion over four hours was determined by the pyloric ligation method and expressed as μmol H⁺ (* p<0.01 when compared to saline, ** p<0.01 when compared to both saline and G-17 infusion)

### REFERENCES

1. Hakanson R. et al., Regulatoiy Peptides 1986;13:225-233.

2. Hakanson R, et al., Scand J Gastroenterol (Suppl). 1991;180:130-136.

3. Ryberg B, et al., Gastroenterology. 1990; 98:33-38.

4. Komorowski R A, and Caya J G. American Journal of Surgical Pathology. 1991; 15:577-585.

5. Helander H F, et al., Scand J Gastroenterol. 1992; 27: 875-883.

6. Lamberts R, et al., Gastroenterology. 1993; 104:1356-1370.

7. Klinkenberg-Knol E C, et al., Ann Intern Med. 1994; 121:161-167.

8. Kuipers E J, et al., Lancet, 1995; 345: 1525-1528.

9. Eissele R, et al., Gastroenterology. 1997; 112:707-717.

10. Levi S, et al., Lancet 1989; 1:1167-1168.

11. Mulholland G, et al., Gut 1993; 34: 757-761.

12. Wang T C, et al., J. Clin. Invest. 1993; 92:1349-1356.

13. Wang T C, et al., J. Clin. Invest. 1996; 98:1918-1929.

14. Wang T C, et al., Gastroenterology. 1998; 114: 675-689.

15. Fox J G, et al., Immunology 1996; 88: 400-406

16. Shay H, et al., Arch. Surg. 1949; 59: 210-213.

17. Fox J G, et al., Gastroenterology 1996; 110:156-166.

18. Schultheis P J, et al., J. Clin. Invest. 1998; 101: 1243-1253.

19. Hakanson R, et al., Histochemistry 1986; 86: 5-17.

20. Romano M, et al., Gastroenterology. 1996; 110: 1448-1455.

21. Dempsey P J, et al., Gastroenterology. 1992; 103: 1950-1963.

22. Murayama Y, et al., Gastroenterology. 1995; 109: 1051-1059.

23. Tsutsui S, et al., Biochem & Biophys Res Comm. 1997; 235: 520-523.

24. Schmidt P H et al., Lab Invest 1999; 79:639-646.

25. Sakagami T, et al., Gut 1996; 39: 639-648.

26. Correa P. Cancer Research 1992; 52: 6735-6740.

27. Li Q, et al., Molecular Endocrinology 1998; 12: 181-192.

28. Watanabe T, et al., Gastroenterology 1998; 115: 642-648.

29. Poynter D, et al., Gut 1986; 27: 1338-1346.

30. Ekman L, et al., Scand J Gastroenterol 1985; 20 (suppl 108): 53-69.

31. Miyazaki Y, et al., Gastroenterology 1999; 116: 78-89.

32. Goldenring J R, et al., Digestive Diseases & Sciences 1996; 41: 773-784.

33. Sharp R, et al., Development. 1995; 121: 149-161.

34. Tamano S, et al., Japanese Journal of Cancer Research. 1995; 86: 435-443.

35. Bayerdorffer E, et al., Gut. 1994; 35:701-704.

36. Falk P, et al., P. N. A. S. USA. 1993; 90: 2035-2039.

37. Danon .J, et al., Gastroenterology. 1995; 108: 1386-1395.

38. McColl K E, et al., Semin Gastrointest Dis 1997; 8: 142-155.

39. El-Omar E M, et al., Gastroenterology. 1997; 113, 15-24.

40. Varro A, et al., J. Biol. Chem. 1990; 265: 21476-21481.

41. Azuma T, et al., Gastroenterology 1987; 93: 322-329.

42. Huebner V D, et al., J Biol Chem. 1991; 266: 12223-12227.

43. Azuma T, et al., J Gastroenterol Hepatol. 1990; 5: 525-529.

44. Higashide S, et al., Am. J. Physiol. 1996; 270 (Gastrointest. Liver Physiol. 33): G220-G224.

45. Dickinson C J, et al., Am J Physiol. 1990; 258 (5 Pt 1): G810-G814.

46. Chen D, et al., Gastroenterology 1999; 116:G0587.

47. Walsh J H. Gastrin. In: Walsh J H and Dockray G J eds. New York:Raven Press, Ltd., 1994:75-121.

48. Black J W, et al., Trends Pharmcol Sci 1987; 8:486-490.

49. Crean G P, et al., Gastroenterology 1969;57:147-155.

50. Majumdar A P N, and Johnson L R. Am. J. Physiol. 1982;242:G135-G139.

51. Koh T J, et al., Gastroenterology 1997; 113:1015-1025

52. Friis-Hansen L, et al., Am J Physiol 1998; 274:G561-G568

53. Wang T C, et al., J. Clin Invest 1996;98:1918-1929.

54. Koh T J, et al., J Clin Invest 1999;103:1119-1126.

55. Palnaes Hansen C, et al., Digestion 1996;57:22-29

56. Matsumoto M, et al., Am J Physiol 1987;252:G315-G319

57. Dickinson C J, et al., Am J Physiol 1990;258:G810-G814

58. Higashide S, et al., Am J Physiol 1996;270:G220-G224

59. Chen D, et al., Regul Pept 1998;169-175.

60. Alumets J, et al., J Physiol (Lond) 1982;323:145-156

61. Shay H, et al., Arch Surg 1949;59:210-213

62. Brown D, et al., Histochem Cell Biol 1996;105:261-267.

63. Chen D, et al., Cell Tissue Res 1196;283:469-478

64. Weibel E R. et al., Int Rev Cytol 1969;26:235-302

65. Weibel E R, and Bolender R P. Stereological techniques for electron microscopic morphometry. In: Hyatt M A, ed. Principle and techniques of electron microscopy. Biological applications. Volume 3. New York:Van Nostrand Reinhold, 1973:237-296.

66. Kaise M, et al., J Biol Chem 1995;270:11155-11160

Equivalents

Those slilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

I claim:

1. A method for characterizing an individual's risk profile of developing duodenal ulcer disease, comprising:
obtaining a ratio of nonamidated gastrins to amidated gastrins in an individual with an elevated total gastrin level,
comparing the ratio of nonamidated gastrins to amidated gastrins to a predetermined value, and
characterizing the individual's risk profile of developing duodenal ulcer disease based upon the ratio of nonamidated gastrins to amidated gastrins in comparison to the predetermined value.

2. The method of claim 1, wherein said individual has not been previously suspected of having duodenal ulcer disease.

3. The method of claim 1, wherein said individual has positive H. Pylori serology.

4. The method of claim 1, wherein said individual is asymptomatic for duodenal ulcer disease.

5. The method of claim 1, wherein the predetermined value is a plurality of predetermined value ranges and said comparing step comprises determining in which of said predetermined value ranges said individual's ratio of nonamidated gastrins to amidated gastrins falls.

6. The method of claim 1, wherein the predetermined value is about 1.1 or higher.

7. The method of claim 1, wherein the predetermined value is about 1.2 or higher.

8. The method of claim 1, wherein the predetermined value is about 1.3 or higher.

9. The method of claim 1, wherein the predetermined value is about 1.4 or higher.

10. The method of claim 1, wherein the predetermined value is about 1.5 or higher.

11. The method of claim 1, wherein the predetermined value is about 1.75, or higher.

12. A method for characterizing an individual's risk profile of developing gastric atrophy leading to gastric cancer, comprising:
obtaining a ratio of nonamidated gastrins to amidated gastrins in an individual with an elevated total gastrin level,
comparing the ratio of nonamidated gastrins to amidated gastrins to a predetermined value, and
characterizing the individual's risk profile of developing gastric atrophy leading to gastric cancer, based upon the ratio of nonamidated gastrins to amidated gastrins in comparison to the predetermined value.

13. The method of claim 12, wherein said individual has positive H. Pylori serology.

14. The method of claim 12, wherein said individual receives proton-pump inhibitor treatment.

15. The method of claim 12, wherein the predetermined value is a plurality of predetermined value ranges and said comparing step comprises determining in which of said predetermined value ranges said individual's ratio of nonamidated gastrins to amidated gastrins falls.

16. The method of claim 12, wherein the predetermined value is about 0.9 or lower.

17. The method of claim 12, wherein the predetermined value is about 0.8 or lower.

18. The method of claim 12, wherein the predetermined value is about 0.7 or lower.

19. The method of claim 12, wherein the predetermined value is about 0.6 or lower.

20. The method of claim 12, wherein the predetermined value is about 0.5 or lower.

21. The method of claim 12, wherein the predetermined value is about 0.4 or lower.

22. A method for evaluating the likelihood that an individual with an elevated total gastrin level will benefit from treatment with an agent useful in treating duodenal ulcer disease, comprising:
obtaining a ratio of nonamidated gastrins to amidated gastrins in the individual,
comparing the ratio of nonamidated gastrins to amidated gastrins to a predetermined value, wherein the ratio of nonamidated gastrins to amidated gastrins in comparison to the predetermined value is indicative of whether the individual will benefit from treatment with said agent, and
characterizing whether the individual is likely to benefit from said treatment based upon said comparison.

23. The method of claim 22, wherein said individual has not been previously suspected of having duodenal ulcer disease.

24. The method of claim 22, wherein said individual has positive H. Pylori serology.

25. The method of claim 22, wherein said individual is asymptomatic for duodenal ulcer disease.

26. The method of claim 22, wherein the predetermined value is a plurality of predetermined value ranges and said comparing step comprises determining in which of said predetermined value ranges said individual's ratio of nonamidated gastrins to amidated gastrins falls.

27. The method of claim 22, wherein the predetermined value is about 1.1 or higher.

28. The method of claim 22, wherein the predetermined value is about 1.2 or higher.

29. The method of claim 22, wherein the predetermined value is about 1.3 or higher.

30. The method of claim 22, wherein the predetermined value is about 1.4 or higher.

31. The method of claim 22, wherein the predetermined value is about 1.5 or higher.

32. The method of claim 22, wherein the predetermined value is about 1.75, or higher.

33. The method of claim 22, wherein the agent useful in treating duodenal ulcer disease is selected from the group consisting of an antacid, a H-2 receptor antagonist, an anticholinergic agent, a coating agent, a prostaglandin, a proton-pump inhibitor, and an antibiotic.

34. A method for evaluating the likelihood that an individual with an elevated total gastrin level will benefit from treatment with an agent useful in treating gastric atrophy, comprising:
obtaining a ratio of nonamidated gastrins to amidated gastrins in the individual,
comparing the ratio of nonamidated gastrins to amidated gastrins to a predetermined value, wherein the ratio of nonamidated gastins to amidated gastrins in comparison to the predetermined value is indicative of whether the individual will benefit from treatment with said agent, and
characterizing whether the individual is likely to benefit from said treatment based upon said comparison.

35. The method of claim 34, wherein said individual is asymptomatic for gastric atrophy disease.

36. The method of claim 34, wherein said individual has positive H. Pylori serology.

37. The method of claim 34, wherein said individual receives proton-pump inhibitor treatment.

38. The method of claim 34, wherein the predetermined value is a plurality of predetermined value ranges and said comparing step comprises determining in which of said predetermined value ranges said individual's ratio of nonamidated gastrins to amidated gastrins falls.

39. The method of claim 34, wherein the predetermined value is about 0.9 or lower.

40. The method of claim 34, wherein the predetermined value is about 0.8 or lower.

41. The method of claim 34, wherein the predetermined value is about 0.7 or lower.

42. The method of claim 34, wherein the predetermined value is about 0.6 or lower.

43. The method of claim 34, wherein the predetermined value is about 0.5 or lower.

44. The method of claim 34, wherein the predetermined value is about 0.4 or lower.

45. The method of claim 34, wherein the agent useful in treating gastric atrophy, is selected from the group consisting of a CCK-B/gastrin receptor antagonist, a proton-pump inhibitor, and a nonamidated gastrin.

46. A method for treating an individual at risk of developing duodenal ulcer disease, comprising:
selecting and administering to an individual having an elevated total gastrin level and an above-normal ratio of nonamidated gastrins to amidated gastrins, an agent selected from the group consisting of an antacid, a H-2 receptor antagonist, an anticholinergic agent, a coating agent, a prostaglandin, a proton-pump inhibitor, and an antibiotic, in an amount effective to inhibit development of duodenal ulcer disease in the individual.

47. The method of claim 46, wherein said individual has not been previously suspected of having duodenal ulcer disease.

48. The method of claim 46, wherein said individual has positive H. Pylori serology.

49. The method of claim 46, wherein said individual is asymptomatic for duodenal ulcer disease.

50. The method of claim 46, wherein the above-normal ratio of nonamidated gastrins to amidated gastrins is about 1.1:1 or higher.

51. The method of claim 46, wherein the above-normal ratio of nonamidated gastrins to amidated gastrins is about 1.2:1 or higher.

52. The method of claim 46, wherein the above-normal ratio of nonamidated gastrins to amidated gastrins is about 1.3:1 or higher.

53. The method of claim 46, wherein the above-normal ratio of nonamidated gastrins to amidated gastrins is about 1.4:1 or higher.

54. The method of claim 46, wherein the above-normal ratio of nonamidated gastrins to amidated gastrins is about 1.5:1 or higher.

55. The method of claim 46, wherein the above-normal ratio of nonamidated gastrins to amidated gastrins is about 1.75:1 or higher.

56. A method for treating an individual at risk of developing gastric atrophy, comprising:
selecting and administering to an individual having an elevated total gastrin level and a below-normal ratio of nonamidated gastrins to amidated gastrins at least one agent selected from the group consisting of a CCK-B/gastrin receptor antagonist, a proton-pump inhibitor, and a nonamidated gastrin, in an amount effective to inhibit development of gastric atrophy in the individual.

57. The method of claim 56, wherein said individual has positive H. Pylori serology.

58. The method of claim 56, wherein said individual receives proton-pump inhibitor treatment.

59. The method of claim 56, wherein said individual is asymptomatic for gastric atrophy.

60. The method of claim 56, wherein said individual has gastric atrophy.

61. The method of claim 56, wherein said individual is asymptomatic for duodenal ulcer disease.

62. The method of claim 56, wherein the below-normal ratio of nonamidated gastrins to amidated gastrins is about 0.9:1 or lower.

63. The method of claim 56, wherein the below-normal ratio of nonamidated gastrins to amidated gastrins is about 0.8:1 or lower.

64. The method of claim 56, wherein the below-normal ratio of nonamidated gastrins to amidated gastrins is about 0.7:1 or lower.

65. The method of claim 56, wherein the below-normal ratio of nonamidated gastrins to amidated gastrins is about 0.6:1 or lower.

66. The method of claim 56, wherein the below-normal ratio of nonamidated gastrins to amidated gastrins is about 0.5:1 or lower.

67. The method of claim 56, wherein the below-normal ratio of nonamidated gastrins to amidated gastrins is about 0.4:1 or lower.

68. The method of claim 56, wherein the nonamidated gastrin is G-Gly or progastrin.

69. A pharmaceutical preparation, comprising:
at least one agent selected from the group consisting of a CCK-B/gastrin receptor antagonist, a proton-pump inhibitor, and a nonamidated gastrin, in an effective amount to inhibit development of gastric atrophy in an individual, and
a pharmaceutically-acceptable carrier.

70. The pharmaceutical preparation of claim 69, wherein the nonamidated gastrin is selected from the group consisting of G-Gly and progastrin.

71. A pharmaceutical preparation, comprising:
a proton-pump inhibitor and a CCK-B/gastrin receptor antagonist, in an effective amount to inhibit development of gastric atrophy in an individual, and
a pharmaceutically-acceptable carrier.

72. The pharmaceutical preparation of claim 71, wherein the proton-pump inhibitor and the CCK-B/gastrin receptor antagonist are in a 1:1 molar ratio.

73. The pharmaceutical preparation of claim 71, wherein the proton-pump inhibitor and the CCK-B/gastrin receptor antagonist are in at least a 2:1 molar ratio.

74. The pharmaceutical preparation of claim 71, wherein the proton-pump inhibitor and the CCK-B/gastrin receptor antagonist are in at least a 3:1 molar ratio.

75. The pharmaceutical preparation of claim 71, wherein the proton-pump inhibitor and the CCK-B/gastrin receptor antagonist are in at least a 4:1 molar ratio.

76. The pharmaceutical preparation of claim 71, wherein the proton-pump inhibitor and the CCK-B/gastrin receptor antagonist are in at least a 10:1 molar ratio.

77. The pharmaceutical preparation of claim 71, wherein the proton-pump inhibitor and the CCK-B/gastrin receptor antagonist are in at least a 20:1 molar ratio.

78. The pharmaceutical preparation of claim 71, wherein the proton-pump inhibitor and the CCK-B/gastrin receptor antagonist are in at least a 30:1 molar ratio.

79. A kit, comprising a package containing:
an agent that selectively binds to a nonamidated gastrin,
an agent that selectively binds to an amidated gastrin,
control epitopes for nonamidated and amidated gastrin, and
instructions for comparing the ratio of nonamidated to amidated gastrins to a predetermined value.

80. The kit of claim 79, further comprising an agent that selectively binds to gastrin.
